(19) United States
(12) Patent Application Publication
(10) Pub. No.: US 2023/0414654 A1
(43) Pub. Date: Dec. 28, 2023

(54) METHODS OF TREATING COPPER METABOLISM-ASSOCIATED DISEASES OR DISORDERS

(71) Applicant: Alexion Pharmaceuticals, Inc., Boston, MA (US)

(72) Inventors: Pan Wei-Jian, Pullman, WA (US); Mark Ma, Madison, CT (US); Brian Meltzer, Boston, MA (US); Eugene Scott Swenson, Madison, CT (US); Scott Edward Mosley, Arlington, MA (US); Ryan Pelto, Middletown, CT (US); Adam Quicquaro, Boston, MA (US); Guillermo del Angel, Lexington, MA (US); Hareesh Chamarthi, Boston, MA (US)

(73) Assignee: Alexion Pharmaceuticals, Inc., Boston, MA (US)

(21) Appl. No.: 18/036,617

(22) PCT Filed: Nov. 12, 2021
(86) PCT No.: PCT/US2021/059239

§ 371 Date: May 11, 2023

Related U.S. Application Data
(60) Provisional application No. 63/113,516, filed on Nov. 13, 2020.
(60) Provisional application No. 63/242,722, filed on Sep. 10, 2021.

(51) Int. Cl. A61K 33/24; A61P 3/00; G01N 33/68

(52) U.S. Cl. CPC A61K 33/24; A61P 3/00; G01N 33/6833; G01N 33/6848

(57) ABSTRACT

This disclosure relates to methods of diagnosing and treating a copper metabolism-associated disease or disorder, such as Wilson disease (WD).

### CROSS-REFERENCE TO RELATED APPLICATIONS

This application claims the benefit of priority of U.S. Provisional Patent Applications No. 63/113,516, filed Nov. 13, 2020, and No. 63/242,722, filed Sep. 10, 2021, which are incorporated herein by reference in their entirety.

### BACKGROUND OF THE DISCLOSURE

### Field of the Disclosure

This disclosure relates to methods of diagnosing and treating a copper metabolism-associated disease or disorder, such as Wilson disease (WD).

### Description of Related Art

Wilson disease (WD) is a rare, autosomal recessive disorder of impaired copper (Cu) transport that results in pathological Cu accumulation. In WD, mutations in the ATP7B gene result in deficient production of adenosine triphosphatase 2 (ATPase2), which in turn leads to impaired biliary excretion of Cu and impaired incorporation of Cu into ceruloplasmin (Cp), a serum ferroxidase, which, in healthy humans, contains greater than 95% of the Cu found in plasma. Consequently, there is an increase of Cu in liver, brain, and other tissues with resultant organ damage and dysfunction. Initial signs and symptoms of WD are predominantly hepatic, neurologic, or psychiatric, but patients often develop combined hepatic and neuropsychiatric disease. Untreated or inadequately treated patients have progressive morbidity, and mortality is usually secondary to hepatic cirrhosis. Other causes of death associated with WD include hepatic malignancy and neurologic deterioration with severe inanition.

The current treatments for WD are the general chelator therapies D-penicillamine and trientine, which chelate Cu and promote urinary Cu excretion, and zinc (Zn), which blocks dietary uptake of Cu through upregulation of intestinal metallothionein. The currently available drugs have high rates of treatment discontinuation due to tolerability and efficacy issues. They require frequent dosing (2 to 4 times per day) and must be taken in a fasted state. Their adverse event (AE) profiles and complicated dosing regimens lead to poor treatment compliance and high rates of treatment failure, a major concern in WD, which requires life-long treatment.

Bis-choline tetrathiomolybdate (“BC-TTM”; BC-TTM; formerly known as WTX101) is an investigational, oral, first-in-class copper-protein-binding molecule being developed for the treatment of WD. BC-TTM has the following structure:

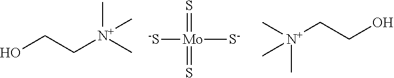

Prior work suggested that BC-TTM improves control of Cu due to rapid and irreversible formation of Cu-tetrathiomolybdate-albumin tripartite complexes (TPCs) leading to rapid de-coppering without mobilization of free Cu that could cause tissue toxicity including neurological deterioration. It is hoped that improved long-term compliance with BC-TTM treatment through improved tolerability and the convenience of a simplified once daily (QD) dosing regimen compared with current therapeutic options could be achieved.

Effective treatment of WD involves establishing and maintaining net negative balance between dietary copper absorption and fecal and urinary copper elimination. Monitoring the effectiveness of copper control often involves periodic measurement of biomarkers in blood and urine. While the “free” copper level can be a conceptual biomarker of disease burden in WD, copper present in blood and urine is believed to be chaperoned by carriers of varying affinity, including ceruloplasmin, metallothionein, albumin, transcuprein, and others. Stabilization or improvement of hepatic, neurologic and psychiatric manifestations is expected to follow copper control, and these factors contribute to the clinician's interpretation of treatment response. Circulating copper in serum or plasma can be assessed through estimation of non-ceruloplasmin-bound copper (NCC), but estimated NCC has limited value because it is an indirect estimate which may generate physiologically and numerically impossible negative NCC results.

### SUMMARY OF THE DISCLOSURE

The disclosure generally provides methods useful for treating a copper metabolism-associated disease or disorder, such as Wilson disease, in a subject.

One aspect of the disclosure provides a method for treating a copper metabolism-associated disease or disorder (such as Wilson disease) in a subject. Such method includes: determining a concentration of total copper and a concentration of labile-bound copper (LBC) in the subject's biological sample; determining the ratio of LBC to total copper in the subject's biological sample; and administering to the subject a therapeutically effective amount of bis-choline tetrathiomolybdate (BC-TTM) when the ratio of LBC to total copper in the subject's biological sample is ≥ between 0.21 and 0.27.

Another aspect of the disclosure provides a method of diagnosing a copper metabolism-associated disease or disorder in a subject, the method comprising: determining a concentration of total copper and a concentration of LBC in the subject's biological sample; determining the ratio of LBC to total copper in the subject's biological sample; and diagnosing the subject with a copper metabolism-associated disease or disorder if the ratio of LBC to total copper in the subject's biological sample is ≥ between 0.21 and 0.27.

Another aspect of the disclosure provides a method of identifying a subject as suited for treatment with bis-choline tetrathiomolybdate, the method comprising: determining a concentration of total copper and a concentration of labile-bound copper (LBC) in the subject's biological sample; determining the ratio of LBC to total copper in the subject's biological sample; identifying the subject as suited for treatment with bis-choline tetrathiomolybdate when the ratio of LBC to total copper in the subject's biological sample is ≥ between 0.21 and 0.27, and optionally administering a therapeutically effective amount of bis-choline tetrathiomolybdate to the subject identified as suited for treatment with bis-choline tetrathiomolybdate.

Another aspect of the disclosure provides a method for treating a copper metabolism-associated disease or disorder in a subject, the method comprising: determining a concentration of total copper and a concentration of directly measured non-ceruloplasmin-bound copper (dNCC) in the subject's biological sample; determining the ratio of dNCC to total copper in the subject's biological sample; and administering to the subject a therapeutically effective amount of bis-choline tetrathiomolybdate when the ratio of dNCC to total copper in the subject's biological sample is ≥ between 0.245 and 0.295.

Another aspect of the disclosure provides a method of diagnosing a copper metabolism-associated disease or disorder in a subject, the method comprising: determining a concentration of total copper and a concentration of dNCC in the subject's biological sample; determining the ratio of dNCC to total copper in the subject's biological sample; and diagnosing the subject with a copper metabolism-associated disease or disorder if the ratio of dNCC to total copper in the subject's biological sample is ≥ between 0.245 and 0.295.

Another aspect of the disclosure provides a method of identifying a subject as suited for treatment with bis-choline tetrathiomolybdate, the method comprising: determining a concentration of total copper and a concentration of dNCC in the subject's biological sample; determining the ratio of dNCC to total copper in the subject's biological sample; identifying the subject as suited for treatment with bis-choline tetrathiomolybdate when the ratio of dNCC to total copper in the subject's biological sample is ≥ between 0.245 and 0.295, and optionally administering a therapeutically effective amount of bis-choline tetrathiomolybdate to the subject identified as suited for treatment with bis-choline tetrathiomolybdate.

In certain embodiments of the methods of the disclosure as described herein, the subject suffers from Wilson disease. In certain embodiments, the subject previously received no treatment for Wilson disease (i.e., a treatment-naïve subject). In certain embodiments, the subject has previously received a standard of care (SoC) treatment for Wilson disease. In certain embodiments of the methods of the disclosure, the subject previously received no treatment for Wilson disease or the subject previously received a standard of care treatment for no more than 4 weeks for Wilson disease.

These and other features and advantages of the claimed invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the compositions and methods of the disclosure, and are incorporated in and constitute a part of this specification. The drawings illustrate one or more embodiment(s) of the disclosure and, together with the description, serve to explain the principles and operation of the disclosure.

FIG. 1A illustrates mean (95% Confidence Interval) of Total Plasma Copper, Labile Bound copper and 24-hour Urine Copper Measured over Time in Study 201 (Full Analysis Set).

FIG. 1B illustrates mean (95% Confidence Interval) of Total Plasma Copper, Labile Bound copper and 24-hour Urine Copper Measured over Time in Study 203 (Full Analysis Set).

FIG. 2 illustrates mean (SD) Plasma Total Molybdenum, Total Copper, Labile-Bound Copper and Ceruloplasmin-Bound Copper Concentration-Time Profiles in Healthy Participants.

FIG. 3 illustrates mean (SD) Plasma Labile-Bound Copper/Total Copper Ratio-Time Profiles in Healthy Participants vs Participants with Wilson disease.

FIG. 4 illustrates the patient populations used in the analysis to determine the LBC/Total Copper ratio (LTC Ratio) optimal threshold value for classification of healthy and Wilson disease patients.

FIG. 5 illustrates boxplots showing the distribution of LTC Ratio for healthy vs Wilson disease patients.

FIG. 6 illustrates an receiver operating characteristic (ROC) curve showing the performance of LTC Ratio for classification of healthy vs Wilson disease patients.

FIG. 7 illustrates the optimal threshold of 0.24 or 24% of LTC Ratio based on f-score yields.

FIG. 8 illustrates the range of LTC Ratio threshold.

FIG. 9 is a boxplot illustrating the distribution of dNCC/Total Copper Ratio (dNCC Ratio) for healthy vs Wilson disease patients.

FIG. 10 is a receiving operating characteristic (ROC) curve illustrating the performance of using dNCC Ratio for classification of healthy vs Wilson disease patients.

FIG. 11 illustrates the optimal threshold of 0.276 or 27.6% of dNCC Ratio based on f-score yields.

FIG. 12 illustrates the range of dNCC Ratio threshold.

FIG. 13 illustrates a Bland Altman scatter plot with the Y axis as the difference between serum and plasma LBC levels in a subject and X the axis as the mean of the two levels.

### DETAILED DESCRIPTION OF THE DISCLOSURE

Before the disclosed processes and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

In view of the present disclosure, the methods and compositions described herein can be configured by the person of ordinary skill in the art to meet the desired need. The present disclosure provides improvements in treating copper metabolism-associated diseases or disorders.

In certain embodiments of the methods of the disclosure as described herein, the copper metabolism associated disease or disorder is Wilson disease.

In certain embodiments, the copper metabolism associated disease or disorder is copper toxicity (e.g., from high exposure to copper sulfate fungicides, ingesting drinking water high in copper, overuse of copper supplements, etc.). In certain embodiments, the copper metabolism associated disease or disorder is copper deficiency, Menkes disease, or aceruloplasminemia. In certain embodiments, the copper metabolism associated disease or disorder is at least one selected from academic underachievement, acne, attention-deficit/hyperactivity disorder, amyotrophic lateral sclerosis (ALS), atherosclerosis, autism, Alzheimer's disease, Candida overgrowth, chronic fatigue, cirrhosis, depression, elevated adrenaline activity, elevated cuproproteins, elevated norepinephrine activity, emotional meltdowns, fibromyalgia, frequent anger, geriatric-related impaired copper excretion, high anxiety, hair loss, hepatic disease, hyperactivity, hypothyroidism, intolerance to estrogen, intolerance to birth control pills, Kayser-Fleischer rings, learning disabilities, low dopamine activity, multiple sclerosis, neurological problems, oxidative stress, Parkinson's disease, poor concentration, poor focus, poor immune function, ringing in ears, allergies, sensitivity to food dyes, sensitivity to shellfish, skin metal intolerance, skin sensitivity, sleep problems, and white spots on fingernails.

As used herein, the terms “treatment” and “treating” mean (i) ameliorating the referenced disease state, condition, or disorder (or a symptom thereof), such as, for example, ameliorating a disease, condition or disorder in an individual who is experiencing or displaying the pathology or symptomatology of the disease, condition or disorder (i.e., reversing or improving the pathology and/or symptomatology) such as decreasing the severity of disease or symptom thereof, or inhibiting the progression of disease; or (ii) eliciting the referenced biological effect.

As provided above, bis-choline tetrathiomolybdate (also known as BC-TTM, ALXN1840, tiomolibdate choline, tiomolibdic acid, and WTX101) is administered in the methods of the disclosure.

BC-TTM is a first-in-class, Cu-protein binding agent in development for the treatment of WD and has been described in detail in International Publication No. WO 2019/110619 (incorporated by reference herein in its entirety). BC-TTM monotherapy has been evaluated in 28 patients with WD, where it was shown that BC-TTM reduced mean serum non-ceruloplasmin-bound Cu (NCC) by 72% at Week 24 compared with baseline. Treatment with BC-TTM was generally well-tolerated, with most reported adverse events (AEs) being mild (Grade 1) to moderate (Grade 2). The most frequently reported drug-related AEs were changes in hematological parameters, fatigue, sulphur eructations, and other gastrointestinal symptoms. Reversible liver function test elevations were observed in 39% of patients; these elevations were mild to moderate, asymptomatic, were associated with no notable increases in bilirubin, and normalized with dose reduction or treatment interruption. No paradoxical neurological worsening was observed upon treatment initiation with BC-TTM.

A therapeutically effective amount of BC-TTM has been previously established. For example, in certain embodiments, BC-TTM may be administered in the range of about 15 to 60 mg per day. In certain embodiments, BC-TTM is administered in an amount of about 15 mg daily. In certain embodiments, BC-TTM is administered in an amount of about 30 mg daily (e.g., about 15 mg taken twice daily or two 15 mg tablets taken once daily). In certain embodiments, BC-TTM is administered in an amount of about 45 mg daily (e.g., about 15 mg taken trice daily or three 15 mg tablets taken once daily). In certain embodiments, BC-TTM is administered in an amount of about 60 mg daily (e.g., about 15 mg taken four times daily or four 15 mg tablets taken once daily).

In certain other embodiments, BC-TTM may be administered in the range of about 15 to 60 mg every other day. In certain embodiments, BC-TTM is administered in an amount of about 60 mg every other day. In certain embodiments, BC-TTM is administered in an amount of about 15 mg every other day. In certain embodiments, BC-TTM is administered in an amount of about 30 mg every other day. In certain embodiments, BC-TTM is administered in an amount of about 45 mg every other day. In certain embodiments, BC-TTM is administered in an amount of about 60 mg every other day.

In certain embodiments of the present disclosure increasing the therapeutically effective amount of BC-TTM during the treatment might provide additional benefits. Thus, in certain embodiments, the therapeutically effective amount of BC-TTM is increased after 6 weeks (i.e., after 42 days) of treatment. For example, in certain embodiments, the initial therapeutically effective amount of BC-TTM (i.e., days 1 to 42) is about 15 mg daily. The increased, subsequent therapeutically effective amount of BC-TTM (i.e., after day 42, such as on day 43 and so on), in certain embodiments, is about 30 mg daily. In certain embodiments, the increased subsequent therapeutically effective amount of BC-TTM is about 45 mg daily. In certain embodiments, the increased subsequent therapeutically effective amount of BC-TTM is about 60 mg daily. For example, in certain other embodiments, the initial therapeutically effective amount of BC-TTM is about 30 mg daily. The increased, subsequent therapeutically effective amount of BC-TTM, in certain embodiments, is about 45 mg daily. In certain embodiments, the increased subsequent therapeutically effective amount of BC-TTM is about 60 mg daily.

In certain embodiments of the present disclosure decreasing the therapeutically effective amount of BC-TTM during the treatment might provide additional benefits. Thus, in certain embodiments, the therapeutically effective amount of BC-TTM is decreased after 6 weeks (i.e., after 42 days) of treatment. For example, in certain embodiments, the initial therapeutically effective amount of BC-TTM (i.e., days 1 to 42) is about 60 mg daily. The decreased, subsequent therapeutically effective amount of BC-TTM (i.e., after day 42, such as on day 43 and so on), in certain embodiments, is about 45 mg daily. In certain embodiments, the decreased subsequent therapeutically effective amount of BC-TTM is about 30 mg daily. In certain embodiments, the decreased subsequent therapeutically effective amount of BC-TTM is about 15 mg daily. For example, in certain other embodiments, the initial therapeutically effective amount of BC-TTM is about 30 mg daily. The decreased, subsequent therapeutically effective amount of BC-TTM, in certain embodiments, is about 15 mg daily.

As used herein, the terms “individual,” “patient,” or “subject” are used interchangeably, and refer to any animal, including mammals, and, in at least one embodiment, humans. In certain embodiments, the subject is a healthy subject. In certain embodiments, the subject suffers from WD. In certain embodiments of the methods of the disclosure as described herein, the subject has cirrhosis. In certain other embodiments, the subject does not have cirrhosis.

The methods of the disclosure are useful as a first line treatment. Thus, in certain embodiments of the methods of the disclosure, the subject previously received no treatment for Wilson disease (i.e., a treatment-naïve subject).

In certain embodiments of the methods of the disclosure, the subject has previously received a standard of care (SoC) treatment for WD. For example, in certain embodiments, the subject has previously received trentine (also known as triethylenetatramine; N-[2-(2-aminoethylamino)ethyl]ethane-1,2-diamine). Trientine may be sold under name CUPRIOR® (GMP-Orphan United Kingdom Ltd), SYPRINE® (Aton Pharma, Inc.), or Cufence (Univar, Inc.). In certain embodiments, the subject has previously received D-penicillamine (also known as penicillamine; (2S)-2-amino-3-methyl-3-sulfanylbutanoic acid). D-penicillamine may be sold under name CUPRIMINE® (Valeant Pharmaceuticals) or DEPEN® (Meda Pharmaceuticals). In certain embodiments, the subject has previously received zinc. In certain embodiments, the subject has previously received trientine, D-penicillamine, and/or zinc. In certain other embodiments, the subject has previously received trientine and/or D-penicillamine.

In certain embodiments of the methods of the disclosure, the subject has received standard of care treatment for WD for no more than 24 weeks. In certain embodiments, the standard of care treatment was no more than 12 weeks, or no more than 6 weeks, or no more than 4 weeks. The standard of care treatment need not be continuous. For example, the subject may receive the treatment on-and-off totaling no more than 24 weeks (e.g., no more than 12 weeks, or no more than 6 weeks, or no more than 4 weeks) of treatment. In certain embodiments, however, the standard of care treatment is continuous.

In certain embodiments of the methods of the disclosure, the subject has received standard of care treatment for WD for no more than 4 weeks.

In certain embodiments of the methods of the disclosure, the subject has received standard of care treatment for WD for at least 4 weeks. In certain embodiments, the standard of care treatment was at least 6 weeks, or at least 12 weeks, or at least 24 weeks, or at least 36 weeks, or at least 48 weeks, or at least 52 weeks long. The standard of care treatment need not be continuous. For example, the subject may receive the treatment on-and-off totaling at least 4 weeks (e.g., at least 6, or at least 12, or at least 24, or at least 36, or at least 48, or at least 50 or at least 52 weeks or at least 103 weeks) of treatment. In certain embodiments, however, the standard of care treatment is continuous.

In certain embodiments of the methods of the disclosure, the subject previously received no treatment or the subject previously received a standard of care treatment for no more than 4 weeks for the copper metabolism-associated disease or disorder, such as for Wilson disease.

In the methods of the disclosure as described herein, the subject completed the standard of care treatment at least 2 weeks prior to administering bis-choline tetrathiomolybdate. In certain embodiments, the subject completed the standard of care treatment at least 3 weeks, at least 4 weeks, or at least 6 weeks prior to administering bis-choline tetrathiomolybdate.

In certain embodiments, when values are expressed as approximations, by use of the antecedent “about,” it will be understood that the particular value forms one possible embodiment and variation of the given value is possible (e.g., about 80 may include 80±10%). It will be further understood that the endpoints of each of the ranges are significant both in relation to the other endpoint, and independently of the other endpoint.

As used herein, “total copper” refers to the sum of all copper species in a biological sample (for example, in whole blood, serum, or plasma). Total copper includes both ceruloplasmin (Cp)-bound copper and all species of non-ceruloplasmin bound copper. In general, total copper may be directly measured with high sensitivity and specificity by mass-spectroscopy, such as inductively coupled plasma-mass spectrometry (ICP-MS).

The term “NCC” refers to the fraction of total copper that is not bound to ceruloplasmin (i.e., “non-ceruloplasmin-bound copper”). Under currently available estimation methods, NCC is estimated using direct measurements of total copper and Cp in the blood (such as, e.g., serum or plasma) and the following formula:

$$NCC [ µM ] = \frac{Total plasma Cu [ µg / L ] - ( 3.15 * ceruloplasmin [ \frac{mg}{L} ] )}{63.5 [ µg / µmol ]} .$$

The calculation is premised on an assumption that six copper atoms are always bound to a single Cp molecule, and that NCC and ceruloplasmin concentrations are directly correlated. In reality, Cp may show considerable heterogeneity in the number of copper atoms associated per Cp molecule. In fact, 6-8 copper atoms can actually bind to Cp, and in WD usually fewer than six copper atoms are associated per Cp molecule. Moreover, clinical data from healthy subjects in Study 108 showed, on average, approximately 4.6 Cu atoms bound per Cp molecule.

Under newly available direct measurement methods, NCC is directly measured using a NCC assay (such directly measured NCC being referred to herein as “dNCC,” and such NCC assay involving direct measurement of NCC being referred to as “dNCC assay” herein). Thus, in certain embodiments disclosed herein, NCC is directly measured using a dNCC assay. For example, in certain embodiments, NCC is directly measured using the dNCC assay described as an “NCC assay” in PCT Patent Application Publication No. WO2021/050850, published on 18 Mar. 2021, herein incorporated by reference in its entirety. In further certain embodiments, the dNCC assay used to directly measure NCC uses the antibodies or antibody mixtures as disclosed in U.S. Provisional Patent Application No. 63/077,155, filed on Sep. 11, 2020, herein also incorporated by reference in its entirety.

In subjects treated with BC-TTM, non-ceruloplasmin-bound copper includes the fraction of total copper that is bound to albumin, transcuprein, and other less abundant plasma proteins (collectively referred to as LBC) or in tetrathiomolybdate-Cu-albumin tripartite complexes (TPCs). The concentration of TPCs cannot be directly measured, but in certain embodiments, the concentration of TPCs may be estimated using molybdenum concentration as a surrogate.

The term “NCC_{corrected}” refers to the fraction of total copper that is not bound to ceruloplasmin or in a TPC (i.e., LBC) and which is calculated by subtracting a direct measure of molybdenum in the blood (such as, e.g., serum or plasma) from the NCC value. “NCC_{corrected}” is thus a correction of the NCC value to account for the presence of molybdenum-copper-albumin tripartite complexes in the blood of BC-TTM-treated subjects.

The terms “LBC” or “labile-bound copper” refer to the fraction of total copper which is bound to albumin, transcuprein, and other less abundant plasma proteins. LBC thus comprises the fraction of total copper which is not bound to either ceruloplasmin or TPCs. In certain embodiments, the LBC fraction is directly measured using an LBC assay. For example, in certain embodiments, LBC is directly measured using the LBC assay described as an “LBC assay” in PCT Patent Application Publication No. WO2021/050850, published on 18 Mar. 2021, herein incorporated by reference in its entirety. In further certain embodiments, the LBC assay used to directly measured LBC uses the antibodies or antibody mixtures as disclosed in U.S. Provisional Patent Application No. 63/077,155, filed on Sep. 11, 2020, herein also incorporated by reference in its entirety. In a biological sample (such as blood, serum, or plasma) in which no TPC is present, the NCC and the LBC fractions are the same.

In certain embodiments, the LTC Ratio is used to identify an optimal threshold for classification of a healthy subject and a copper metabolism-associated disease or disorder subject. In certain embodiments, the LTC Ratio optimal threshold is between 0.21 and 0.27. In certain embodiments, the LTC Ratio optimal threshold is 0.24. For example, the LTC Ratio can be used in methods of diagnosis of a copper metabolism-associated disease or disorder. In certain embodiments, a LTC Ratio ≥ between 0.21 and 0.27 in the subject's biological sample (such as, e.g., blood, serum, or plasma) indicates the subject has a copper metabolism-associated disease or disorder. In certain embodiments, a LTC Ratio ≥0.24 in the subject's biological sample indicates the subject has a copper metabolism-associated disease or disorder.

In certain embodiments, the dNCC Ratio is used to identify an optimal threshold for classification of a healthy subject and a copper metabolism-associated disease or disorder subject. In certain embodiments, the dNCC Ratio optimal threshold is between 0.245 to 0.295. In certain embodiments, the dNCC Ratio optimal threshold is 0.276. For example, the dNCC Ratio can be used in methods of diagnosis of a copper metabolism-associated disease or disorder. In certain embodiments, a dNCC Ratio ≥ between 0.245 and 0.295 in the subject's biological sample (such as, e.g., blood, serum, or plasma) indicates the subject has a copper metabolism-associated disease or disorder. In certain embodiments, a dNCC Ratio ≥0.276 in the subject's biological sample indicates the subject has a copper metabolism-associated disease or disorder.

In certain embodiments, an effective amount can be an amount suitable for:
(i) inhibiting the progression of the disease;
(ii) prophylactic use for example, preventing or limiting development of a disease, condition or disorder in an individual who may be predisposed or otherwise at risk to the disease, condition or disorder but does not yet experience or display the pathology or symptomatology of the disease;
(iii) inhibiting the disease; for example, inhibiting a disease, condition or disorder in an individual who is experiencing or displaying the pathology or symptomatology of the disease, condition or disorder;
(iv) ameliorating the referenced disease state, for example, ameliorating a disease, condition or disorder in an individual who is experiencing or displaying the pathology or symptomatology of the disease, condition or disorder (i.e., reversing or improving the pathology and/or symptomatology) such as decreasing the severity of disease; or
(v) eliciting the referenced biological effect.

### Example

The methods of the disclosure are illustrated further by the following Example, which is not to be construed as limiting the disclosure in scope or spirit to the specific procedures and compounds described therein.

### BACKGROUND

The concentration of total copper in blood has generally been considered uninformative for monitoring treatment response in WD. The total copper concentration in serum or plasma is highly dependent on the concentration of ceruloplasmin, which is known to be abnormally low in most patients with WD due to the inherited functional deficiency of ATP7B. Therefore, patients with WD have a paradoxically low level of total copper in serum or plasma. Successful treatment may be expected to drive the total copper concentration even lower, which is challenging to interpret. The optimal target concentration of total copper in the blood of a treated WD patient is unknown, but likely falls well below laboratory reference ranges established among healthy individuals with normal ceruloplasmin.

At the time that Study 203 (in participants with WD treated with SoC) and Study 201 (in participants with WD treated with BC-TTM) were initiated, NCC and NCC_{corrected}, respectively, were selected as the surrogate primary efficacy outcome measures. The rationale was that NCC represents the loosely bound or “exchangeable” fraction of copper, which accounts for the organ damage in WD. Urinary copper excretion was not expected to change with BC-TTM treatment, therefore measuring the change in 24-hour urinary copper excretion was not expected to be informative.

Phase 2 clinical samples were re-analyzed from the following studies using the newly developed and technically-validated LBC assay to directly quantify plasma “free” NCC or NCC_{corrected} when BC-TTM is administered:
Study 201, an open-label efficacy and safety study of BC-TTM in patients with WD, and
Study203, a natural history/standard of care study in patents with WD.

An integrated analysis of plasma total molybdenum, total copper, NCC, and LBC (LBC ultimately replacing NCC_{corrected}) in participants treated with BC-TTM or standard of care revealed previously unrecognized dynamic changes during BC-TTM treatment in both total copper and LBC. These dynamic changes in total copper and LBC were not observed with SoC treatment. The key characteristics of each SoC treatment, as well as BC-TTM, are presented in Table 1.

TABLE 1
Comparison of BC-TTM to Approved WD SoC Therapies

| Parameter | BC-TTM | Penicillamine | Trientine | Zinc |
|---|---|---|---|---|
| Classification | Albumin-Cu | Cu-Chelator | Cu Chelator | Enterocyte |
| | binding agent | | | metallothionein |
| | | | | inducer |
| Cu-Binding | 19.6 | 15.6 | 15.8 | NA |
| Affinity | | | | |
| (−log₁₀[Kd]) | | | | |
| Cu-Binding | Cu⁺ and Cu²⁺* | Cu⁺, Cu²⁺, and | Cu²⁺, and other | NA |
| Selectivity | only, | other divalent | divalent cations; | |
| | not Zn²⁺, Fe²⁺ | cations; need | need metal | |
| | Ca²⁺, Mg²⁺, or | metal | supplements | |
| | Mn²⁺ | supplements | | |
| Drug:Cu- | 1:1, 1:2, 1:3, 1:4, | 2:1 | 1:1 | NA |
| Binding Molar | 1:6 | | | |
| Ratio | | | | |
| Binding to Cu | Yes, including | Yes, Cu in | No | No |
| in hepatic | metallothionein- | lysosomes of | | |
| tissue | Cu | hepatocytes and | | |
| | | Kupffer cells, but | | |
| | | not | | |
| | | metallothionein- | | |
| | | Cu | | |
| Binding to Cu | Yes, forms stable | Yes | Yes | No |
| in the blood | TPCs with | | | |
| | covalent bonds | | | |
| Impact on | None; Cu | May damage | Does not cross | No |
| CNS | mobilization after | blood-brain | blood-brain barrier | |
| | BC-TTM | barrier by | | |
| | treatment does | increased free Cu | | |
| | not redistribute to | and oxidative | | |
| | the brain tissue | damage in brain | | |
| Mobilization | Yes (Study 106; | No (Study 203) | No | No (Study 203) |
| of Copper | Study 201) | | (Study 203) | |
| from Tissue | | | | |
| Route of | Biliary/fecal | Excreted in the | Excreted in the | Non-absorbed |
| Copper | excretion of TPCs | urine | urine | dietary copper |
| Elimination | | | | excreted in feces |

### Study 201

Study 201 was a Phase 2, multicenter, open-label study to evaluate the efficacy and safety of BC-TTM, when administered for 24 weeks, in newly diagnosed participants with WD aged 18 years and older, with an extension phase of 36 months. This study was conducted in US and European sites. Participants received BC-TTM at individualized doses between 15 and 120 mg/day. A total of 28 participants were enrolled and treated (10 in Cohort 1 [treatment experienced] and 18 in Cohort 2 [treatment naïve or minimally treated]). One participant was enrolled in Cohort 2 but not treated.

The primary objective of the study was to evaluate the efficacy of BC-TTM for 24 weeks on NCC levels adjusted for molybdenum plasma concentration in participants with WD aged 18 and older. NCC levels within or above the normal reference range at enrollment were required. The secondary objectives included safety and tolerability, effects of BC-TTM on hepatic measures, disability, and neurological status, and collection of PK data. The 36-month extension phase evaluated the tolerability and long-term safety and efficacy of BC-TTM. Dosing was individualized, guided by NCC levels (adjusted for molybdenum plasma concentration) and safety data. The primary efficacy endpoint was related to the proportion of successful participants, i.e., the proportion of participants who achieved plasma “free” copper control or improvement from baseline. In a post-hoc analysis, plasma copper control was also evaluated using results from the LBC assay to quantify “free” copper in plasma that was not bound to either ceruloplasmin or the TPC.

Over the course of Study 201, the maximum allowed dose of BC-TTM was decreased in order to manage initially observed drug-related liver enzyme elevations. Participants received a starting dose of BC-TTM of 15 to 120 mg/day based on baseline NCC concentrations for the first 4 to 8 weeks, with subsequent response-guided individualized dosing over the rest of the study period. Although dosing of BC-TTM was initially given twice daily (BID), a protocol amendment in March 2016, before most enrollment had taken place, implemented once daily dosing (if deemed appropriate by the investigator). After a significant treatment-emergent elevation of alanine aminotransferase (ALT) in a participant receiving 120 mg/day, the dose regimen was amended from a maximum dose of 300 mg/day to 60 mg/day. Due to small sample size and data variability, the median values for the weighted averages of the doses will be cited throughout.

The primary endpoint in study 201 was the change in NCC/NCC_{corrected} from baseline to Week 24. Treatment with BC-TTM resulted in rapid reductions in “free” copper concentrations as measured/calculated by NCC_{corrected} from Week 4 (p=0.0199). Mean reductions in NCC concentration were also statistically significant at Week 24 vs. baseline (p<0.0001). By Week 24, almost all participants were considered to be successfully treated with BC-TTM, regardless of previous time on treatment (90.0% of participants included in Cohort 1 and 83.3% of participants included in Cohort 2). These results were sustained throughout the long-term Extension Period; at the time of the last assessment, 24 (85.7% [9 (90.0%) in Cohort 1 and 15 (83.3%) in Cohort 2]) were considered successful in relation to treatment with BC-TTM according to the prospectively defined study primary endpoint.

### Study 203

Study 203 was a 24-month multicenter, observational study to assess copper parameters in participants with WD treated with SoC medications (the chelators penicillamine and trientine, and zinc). Depending on the duration of prior WD treatment, participants were allocated to two cohorts (Cohort 1: prior WD treatment of >28 days; Cohort 2: treatment naïve or prior WD treatment of ≤28 days). Participants in the study continued with their existing medication and did not receive BC-TTM or any other investigational drug.

The primary objective of the study was to assess plasma and urine copper parameters in participants with WD treated with SoC. The secondary objectives of this study were to compare copper parameters with corresponding clinical data, including medical and medication history, clinical laboratory results, WD medications, and Clinical Global Impression (CGI). The primary efficacy endpoint was the proportion of participants who achieved or maintained normalized concentrations of NCC (0.8 μM-2.3 μM) or reached a reduction of at least 25% in NCC during 6 months of treatment if above the reference range at the time of enrollment. In a post-hoc analysis, plasma copper control was also evaluated using results from the LBC assay to quantify copper in plasma that is not bound to ceruloplasmin.

A total of 64 participants were enrolled, of which 57 completed the study and 7 discontinued. A total of 33 (51.6%) participants were successful at achieving and/or maintaining the normal reference range for NCC through Month 6.

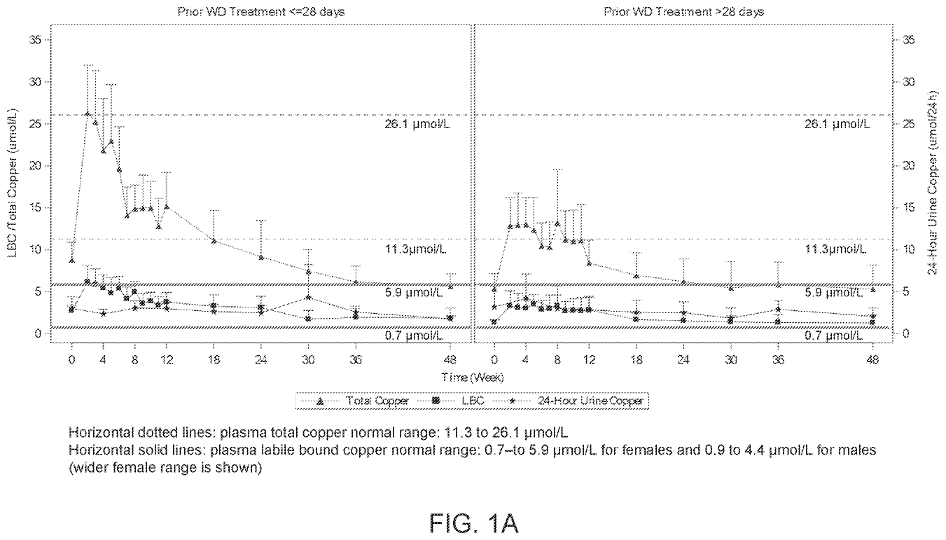

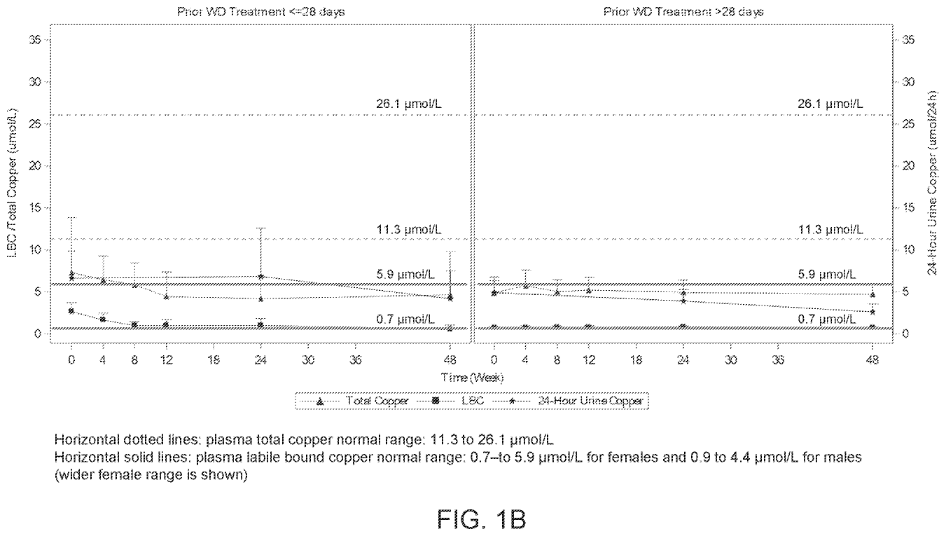

The primary efficacy analysis was repeated with LBC data. Overall, 62 (96.9%) of participants were successful at achieving and/or maintaining LBC within the reference range (male: 0.9-4.4 μmol/L; female: 0.7-5.9 μmol/L) through Month 6. The analyses of Study 203 plasma samples reveal minimal mobilization of copper from tissues (FIG. 1B), in comparison to that in Study 201 (FIG. 1A).

Blood sampling was less frequent in study 203, but the difference in trends compared with Study 201 is very clear. There was a very small reduction in total copper and LBC in Cohort 2 (treatment-naïve) participants between baseline and Week 4, consistent with a modest decoppering effect in plasma, without appreciable mobilization from tissue. Thereafter, there was no change in either total copper or LBC. Among treatment-experienced participants, both total copper and LBC were low at baseline with virtually no change at all throughout 48 weeks. While baseline 24-hour urine copper excretion was numerically greater in Cohort 2 (left panel), the 24-hour urine copper excretion changed minimally over time, with a small decrease noted at Week 48 in Cohort 2 treatment-naïve participants.

By Week 48, the total copper concentration reached a nadir of approximately 4 μM. While this is below the normal reference range, it is to be expected that participants with WD will almost always fall well below the reference range for total copper due to the relative deficiency in ceruloplasmin. As noted above, the normal reference range for total copper does not apply in WD.

In these participants treated with penicillamine, trientine and/or zinc, 51.6% of participants achieved “success” as defined by achieving a target NCC concentration within the reference range by the end of 6 months. In the post-hoc analysis, 96.9% of participants achieved success as defined success by achieving a target LBC concentration within the reference range by the end of 6 months. It is not clear if either measure is informative with regard to copper control or clinical manifestations of WD. Measuring the change (or percent change) from baseline to Week 48 in plasma NCC, total copper or LBC concentration also provided little insight into copper control. In contrast, the striking copper mobilization effect of BC-TTM during the first 24 weeks of treatment in Study 201 makes this difference very clear.

### Study 106

Study 106 was an open-label, 2-period, parallel group, Phase 1 study to assess whether single-dose BC-TTM PK and safety are comparable between healthy Japanese and non-Japanese participants. It was conducted at a single center in the UK to support the enrollment of Japanese patients in the ongoing Phase 3 Study 301 and the New Drug Application of BC-TTM in Japan.

Twenty-four participants were enrolled (12 Japanese and 12 non-Japanese). During Dosing Period 1, participants received a single dose of one 15 mg BC-TTM EC tablet at Hour 0 on Day 1 following an overnight fast. During Dosing Period 2, participants received a single dose of 4×15 mg (for a total of 60 mg) BC-TTM EC tablets at Hour 0 on Day 1 following an overnight fast. Participants were followed 10 days after dosing within each Dosing Period, and a minimum of 14 days (+2 days) separated the dosing periods. PK and PD concentrations were measured over the next several hours for the first day and once daily for the next 9 days. Therefore, Study 106 permits examination of PK and PD profile changes within hours after drug administration. These PK/PD profiles can be compared with the 12-hour dense PK/PD profiles obtained in Study 201 on Day 1 and at Weeks 12 and 24 visits.

### DISCUSSION

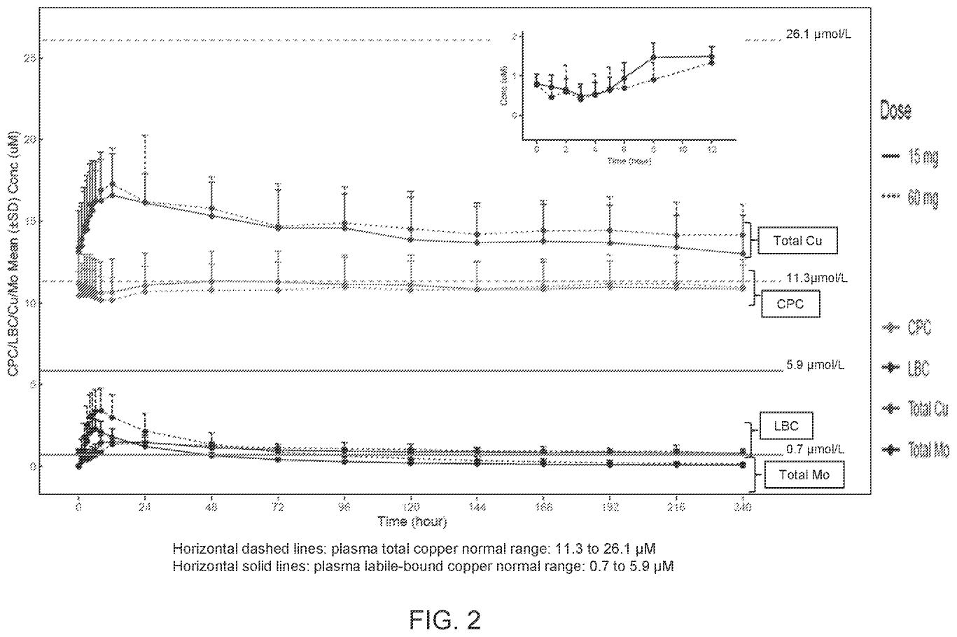

When BC-TTM PK parameters and PD profiles were compared between Japanese and non-Japanese participants, there were no significant differences after a single oral dose of 15 mg or 60 mg of BC-TTM under fasting conditions. Therefore, the ethnic cohorts were combined for the purpose of the analyses in this document. FIG. 2 presents the mean (SD) PK/PD profiles in healthy participants from Study 106. Details of these data presentation and other information can be found in the CSR for Study 106.

Assessments have been conducted for Study 106 and compared with Study 201 to aid in the selection of therapeutic monitoring tools for treating participants with WD with BC-TTM. This may be accomplished and confirmed with PK/PD data such as plasma total molybdenum, total copper, LBC and LBC/total copper ratio, ceruloplasmin-bound copper (CpC), and TPC from Study 201 to assure participants with WD are not under-treated or over-treated when transitioning with a dose titration from de-coppering phase to maintenance phase. Due to disease and participant heterogeneity and the corresponding implementation of individualized dosing in the BC-TTM clinical development program, each participant may respond to the treatment differently, time-wise and magnitude-wise. Therefore, the proposed investigation into the comprehensive and/or combinations of the PK/PD parameters noted above are critical in defining the time frame and parameter range for achieving optimal efficacy and safety treatment outcomes in participants with WD.

Total molybdenum: Following either the single 15 mg or the single 60 mg dose of BC-TTM, plasma concentration-time profiles of total molybdenum were comparable, as were PK parameters. The time to maximum plasma molybdenum (Tₘₐₓ) was 5 hours and the estimated t_{1/2} was 64-84 hours, or 2.7-3.5 days. Plasma total molybdenum Cₘₐₓ, and AUC increased less than dose-proportionally from 15 mg to 60 mg and a dose-proportional (linear) increase corresponding to the higher dose of BC-TTM was not observed (FIG. 2). Based on mean dose-normalized plasma total and ultrafiltrate molybdenum AUCs, approximately 96% of the plasma molybdenum was anticipated to be in the form of TPCs.

Total copper: The combined ethnicity data had a plasma total copper mean of 13.1 μM (range: 6.2 to 19.2 μM) at predose baseline. The maximum individual plasma total copper concentrations of 23.0 and 21.7 μM were observed after single 15 mg and 60 mg doses of BC-TTM, respectively. These values are within the reference range for normal healthy subjects' serum or plasma total copper, which has been reported to be between 11.3-26.1 μM by using an ICP-MS assay (LabCorp. copper, serum or plasma [internet]. 2020a. [cited 16 Jul. 2020]. Available from: https://www.labcorp.com/tests/001586/copper-serum-or-plasma).

Plasma total copper concentrations reached their maximum 8-12 hours post dose, with a maximum median increase from baseline of approximately 26-34% (FIG. 2). After the 8 to 12-hour post dose time point, copper concentrations gradually decreased with the median percent change from baseline reaching within approximately <10% of the predose baseline. At 240 hours post dose, total copper concentrations had returned to predose baseline.

The early increase in plasma total copper closely paralleled the appearance of total molybdenum, consistent with rapid formation of TPC almost immediately after absorption of BC-TTM. The maximum plasma concentration of total copper followed the maximum plasma concentration of total molybdenum by approximately 3-7 hours after dose.

LBC and LBCtota copper ratio: The combined ethnicity data had a plasma LBC mean of 0.805 μM (range: 0.48 to 1.49 μM) at predose baseline. The average plasma LBC concentration from all participants at baseline was approximately 6% (±2%) of the plasma total copper concentration (LBC/total copper ratio). There was an initial 3-hour transient decrease (while plasma total molybdenum was increasing) and then increase (FIG. 2 insert), with eventual return to pre-dose baseline concentrations of plasma LBC over time after either 15 mg or 60 mg dose (FIG. 2). The maximum individual plasma LBC concentrations of 2.79 and 2.93 μM were observed after single 15 mg and 60 mg BC-TTM, respectively. These values are within the reference range for normal healthy subjects' plasma LBC (see Table 2).

TABLE 2
Reference Ranges for Labile-Bound Copper

Labile-Bound-Copper (μmol/L)

| Sex | LLN | ULN |
|---|---|---|
| Female | 0.7 | 5.9 |
| Male | 0.9 | 4.4 |

Median plasma LBC concentrations from the combined ethnicity and dose data reached their nadir of approximately 50-70% pre-dose baseline concentration at 3 hours post dose and subsequently increased to approximately 80-90% of pre-dose baseline concentration at 12-24 hours post dose and returned to baseline 10 days after dose (FIG. 2). The initial decrease in plasma LBC for the first 3 hours while plasma total molybdenum was still increasing toward Cm, implies that BC-TTM had the initial capacity of reducing plasma “free” copper or LBC. The subsequent increase in LBC may be related to the overall liver decoppering or copper mobilization effects of BC-TTM with maximum plasma total copper and LBC concentrations reached at approximately the same time of about 12 hours post-dose. It is possible that, during the initial 12-hour time window after dosing, relatively small amount of mobilized copper molecules could not be fully captured in the stable TPCs and thus became measurable as LBC. It is possible that BC-TTM binds to Cu when it resides in a very specific position such as N-terminal histidine in the albumin molecule, such that, copper molecules bound anywhere else may not bind with BC-TTM and form TPCs. Another possibility is that we have data showing transcuprein level increased in post-dose samples from Study 106 within 12-24 hours of BC-TTM treatment, which may also explain the peak of time of plasma LBC concentration.

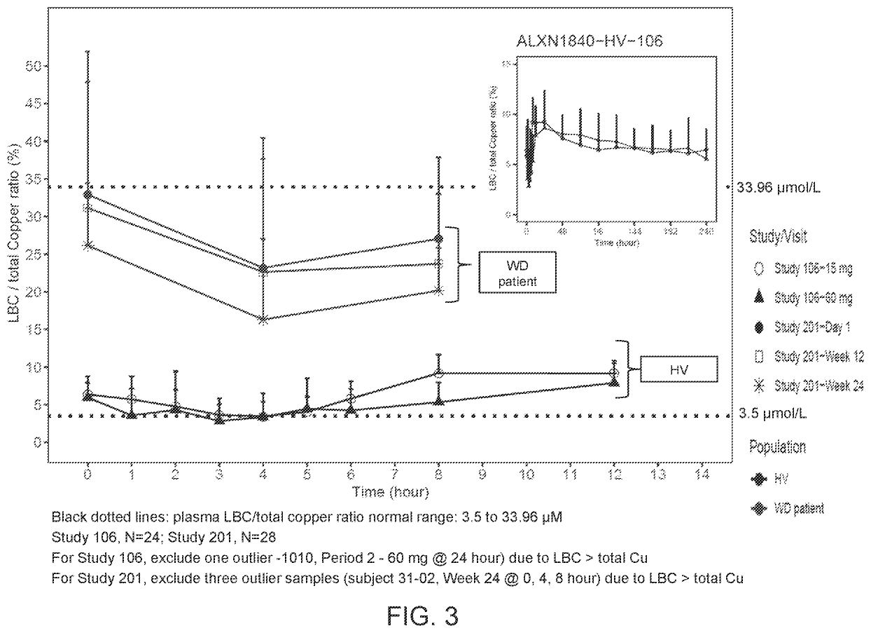

Study 106 mean (SD) plasma LBC/total copper ratio-time profiles show similar trend to plasma LBC profiles (FIG. 3, insert). The AUEC calculations based on area under the effect (plasma total copper and LBC)-time curves after BC-TTM administration (all participant data pooled, both ethnicities and doses) show that the percentage of plasma LBC remains virtually unchanged at 7% (±1%) relative to the plasma total copper. This suggests that, even though there were dynamic changes in both plasma total copper and LBC concentrations due to tissue copper mobilization, the relative amount of “free” copper remained virtually unchanged and was also maintained well within the normal reference range after a single dose of BC-TTM.

In Study 201, 12-hour dense PK/PD samples for plasma total molybdenum and copper were collected on Day 1, Week 12, and Week 24. Plasma samples for assessing “free” copper were also collected at 0, 4, and 8 hours during those visits and assayed for LBC concentrations. For the purpose of comparison across Studies 106 and 201, FIG. 3 has super-imposed LBC/total copper ratios-time profiles within the 12-hour time window after dosing from both studies. All of those who contributed to Cₘₐₓ are used to conduct a weighted average dose calculation: for Day 1, Week 12, and Week 24, the corresponding numbers are 30 mg, 33.6 mg, and 35.8 mg. FIG. 3 (top) shows that the mean plasma LBC/total copper ratio-time profile on Day 1 pre-dose was just below the upper bound of the normal range with SD extending well above normal. After a weighted average dose of 30 mg BC-TTM, LTC Ratio dropped ˜30% from baseline at 4 hours post dose with slight rebound after 8 hours. After 12 weeks of repeated dosing and under a weighted average dose of 33.6 mg BC-TTM treatment on the day of visit, mean LBC/total copper ratio-time profile dropped slightly vs. Day 1, indicating decoppering phase may be still on-going. At Week 24, pre-dose LTC Ratio had substantial reduction of ˜30% and a nadir of ˜50% drop at 4 hours post-dose compared to Day 1 pre-dose baseline. The weighted average dose of BC-TTM increased slightly to 35.8 mg on the day of visit at Week 24, indicating that the decoppering phase may have been completed, even though the overall LBC/total copper ratio-time profiles were still above those from Study 106 after a single dose of 15 mg or 60 mg BC-TTM (FIG. 3, bottom).

Clinical evidence supports the use of the LTC Ratio or the dNCC Ratio for the diagnosis of participants with WD. For example, dNCC Ratio could be used for this purpose, because in subjects in which no TPC is present, i.e., subjects not being treated with BC-TTM, the NCC and LBC fractions are the same. While not being bound by a theory, it is believed that LBC (or dNCC in subjects not being treated with BC-TTM) represents the fraction of circulating copper bound to albumin and proteins other than the copper specific proteins, Cp and transcuperin. The LBC value (or dNCC value in subjects not being treated with BC-TTM), when expressed as a percentage of or in a ratio relative to total copper, reflects altered copper metabolism in untreated WD patients, and has the potential to be diagnostic of WD with a single blood draw. For example, by comparing to an established normal reference range for the healthy patients, an elevated LTC Ratio value or dNCC Ratio may be used as a standalone test to diagnose WD or identify a subject as suited for treatment with BC-TTM.

Based on meta-analysis, LTC Ratio mean and range have been calculated based on Study 106, Study 104, Study 201, Study 203, Study 108, and Study 301 data. This data is presented in Table 3.

TABLE 3
Comparison of plasma LTC Ratio in different subjects

| | Ratio of LBC (μmol/L) to Total Copper (μmol/L) (%) | | |
|---|---|---|---|
| | WD subjects, prior treatment | | |
| Study | Naïve | Experienced | Non-WD (healthy) subjects |
| Study 201 | 34.80 (n = 16) | 32.79 (n = 9) | — |
| Study 203 | 34.33 (n = 6) | 23.99 (n = 40) | — |
| Study 104^{a} | — | — | 4.8 (n = 48) |
| Study 106 ^{b} | — | — | 6.4 (n = 24) |
| Study 301 ^{c} | 35.3 (n = 35) | 29.9 (n = 164) | — |
| Study 108 ^{d} | — | — | 3.7 (n = 17) |
| Reference ^{e} | — | — | 12.5 (n = 248) |
| Weighted Mean | 35.06 (n = 57) | 28.91 (n = 213) | 10.52 (n = 337) |
| Ranges | 5.3-76.5 | 1.7-96.3 | 2.0-33.96 |

^{a}Study 104 is a phase 1 clinical study registered with EudraCT under study No. 2019-000516-28 aimed to assess the relative bioavailability of BC-TTM oral formulation. The value was obtained using the experimental and statistical methods noted below. Mean (SD): 4.81 (1.968); Median (IQR): 4.45 (1.61); Q1, Q3: 3.52, 5.14; Min, Max: 2.9, 13.8; and 95% CI: (4.24, 5.38)
^{b} The value was obtained using the experimental and statistical methods noted below. Mean (SD): 6.37 (2.380); Median (IQR): 5.84 (2.02); Q1, Q3: 4.92, 6.94; Min, Max: 3.2, 14.6; and 95% CI: (5.37, 7.38).
^{c} Study 301 is a phase 3 clinical study registered under study No. NCT03403205 aimed to evaluate the efficacy and safety of BC-TTM administered for 48 weeks versus standard of care in Wilson disease patients aged 12 years and older.
^{d} Study 108 is a phase 1 clinical study registered under study No. NCT04594252 aimed to assess the copper balance in patients following administration of BC-TTM.
^{e} The values was obtained using the experimental and statistical methods noted below.

Methods used to obtain values In Table 3: The “Reference” samples in Table 3 were individual lots of adult human lithium heparn plasma sample purchased from BioIVT and Lampire Biological Laboratories. Plasma samples were stored at −70° C. upon arrival. The concentration of ceruloplasmin-bound copper (CpC) and LBC in plasma samples was determined using a validated ICP-MS method. In the assay, Cp was first immunocaptured using an anti-Cp antibody and CpC concentration was measured by ICP-MS method. On the resulting non-ceruloplasmin solution, EDTA chelation followed by filtration were performed to isolate LBC from plasma for ICP-MS analysis. The total serum copper concentration is made up of CpC and LBC.

The ratio of LBC to total copper was expressed as a percentage and calculated as

$$\% LBC = \frac{LBC}{total copper} \times 100.$$

Means and variances were compared between genders. The assumption of normality was tested using the Kolmogorov-Smirnov and Anderson-Darling tests. The Box-Cox method was then applied to identify a normalizing transformation. Outlier detection was performed on the transformed data using the Dixon and Tukey methods. Gender means and variances were compared on the transformed data, and the data were partitioned based on the results of a z-test. Subgroup reference intervals were obtained using the nonparametric method as suggested in Clinical and Laboratory Standards Institute (2008) (EP28-A3c: Defining, Establishing, and Verifying Reference Intervals in the Clinical Laboratory; Approved Guideline (3rd edition), Wayne, PA).

Study populations for “Reference” Intervals In Table 3: The samples for the “Reference” intervals provided in Table 3, above, consisted of LBC and total copper concentrations expressed in ng/mL from 122 female and 126 male subjects. The median age of the subject was 40 years (X=41.3; range 18-74). The median age for females was 39.0 years (X=40.6; range 18-74) and the median age for males was 42.0 years (X=41.9; range 18-70). Descriptive statistics of % LBC overall and by gender are shown in Table 4. A t-test revealed that % LBC means were different between genders (X=10.03% for females, X=14.97% for males; p<0.0001). The group variances were also unequal (folded F-test, p=0.0008).

TABLE 4
Plasma LTC Ratio in reference population

| | | | | | | | Reference Interval (%) | | 90% Cl | |
|---|---|---|---|---|---|---|---|---|---|---|
| | | Mean | Std. | Std. | Min. | Max. | 2.5th | 97.5th | 2.5th | 97.50 |
| | N | (%) | Dev | Err | (%) | (%) | Percentile | Percentile | Percentile | Percentile |
| All Subjects | 248 | 12.54 | 6.683 | 0.424 | 2.95 | 45.34 | | | | |
| Female | 122 | 10.03 | 5.208 | 0.472 | 2.95 | 25.88 | 3.50 | 22.65 | 2.95-4.00 | 21.66-25.88 |
| Male | 126 | 14.97 | 7.066 | 0.629 | 3.66 | 45.34 | 6.73 | 33.96 | 3.66-8.22 | 31.00-45.34 |

### Calculated LTC Ratio Optimal Threshold Value for Classification of Healthy and Wilson Disease Patients

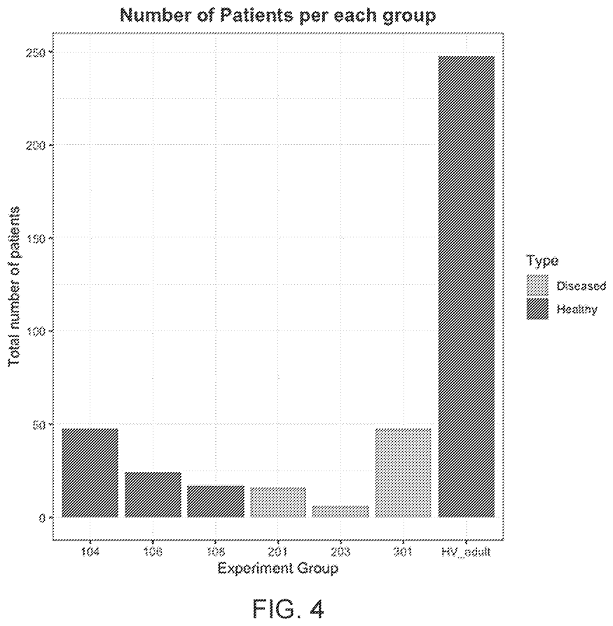

To determine the optimal threshold to classify a patient as healthy or as having Wilson disease using LTC Ratio, the LTC Ratio of 337 healthy patients and 70 Wilson disease patients was analyzed (FIG. 4). Specifically, 248 “Reference” samples (identified as “HV” in FIG. 4) and 89 healthy patient samples from Study 104, Study 106 and Study 108 were analyzed. The 70 Wilson disease patients' cohort is from Study 301, Study 201, and Study 203. The analysis focused on treatment of naïve patients due to potential confounding effects by previous treatment.

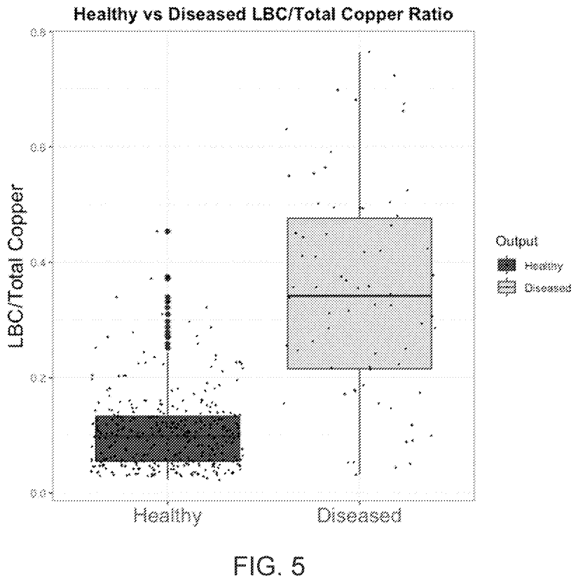

The distribution of healthy vs Wilson disease patients were represented in boxplots (FIG. 5). Using boxplots, the distribution of numerical data and skewness was observed through displaying averages. A T-test was performed to show the significance (p-value <2.2e-16) in the difference of mean of LTC Ratio between healthy and Wilson disease patients. The performance at each LTC Ratio data was measured between 0-1 to classify healthy and Wilson disease patients.

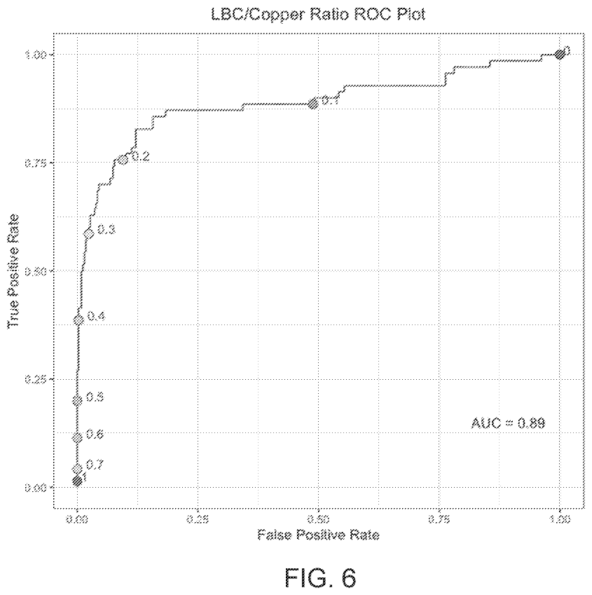

ROCR (Sing T, et al. (2005) ROCR: visualizing classifier performance in R. Bioinformatics 21(20):3940-1) was used, which is a R package to evaluate and a ggplot R package to visualize the performance of this scoring classifier (FIG. 6). A receiving operating characteristic (ROC) curve was plotted (FIG. 6) showing the performance of using LTC Ratio for classification of healthy vs Wilson disease patients. Because the datasets were slightly unbalanced, the F-score was used to define the optimal threshold. In addition, the ROCR tool (Sing T et al. (2005)) was used to measure F-score.

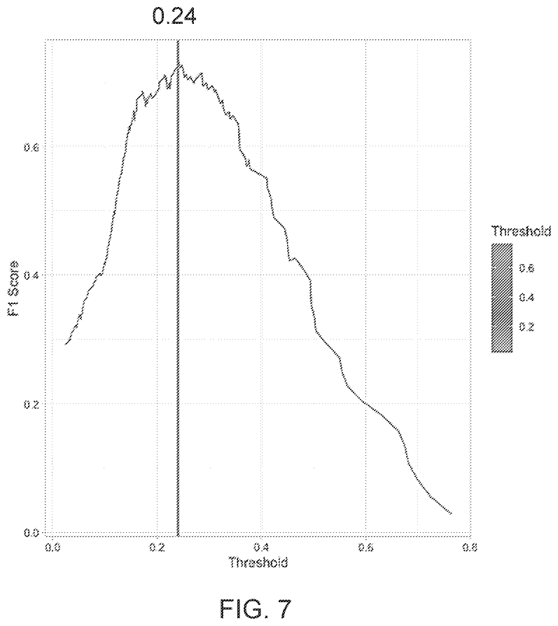

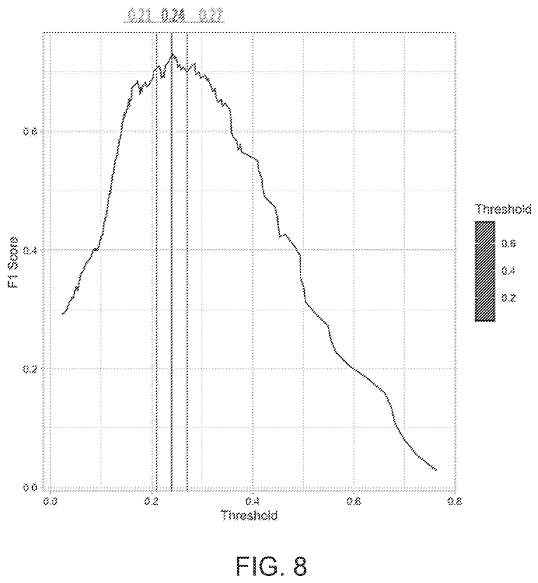

With a F-score maximum at ˜0.73 the optimal threshold of 0.24 or 24% of LTC Ratio was observed (FIG. 7). Considering the specificity and sensitivity trade-offs, a range of thresholds with a F-score above 0.7 were investigated. A range for the LTC Ratio threshold was defined as shown in FIG. 8 (i.e., from 0.21 to 0.27, inclusive). Finally, the cross-tabulation of observed and predicted classes with associated statistics with confusionMatrix function from Caret (Kuhn. (2008), J. Stat. Soft., Building predictive models in R using caret Package 28; 1-26) library in R was calculated.

Calculated dNCC Ratio Optimal Threshold Value for Classification of Healthy and Wilson Disease Patients

To determine the optimal threshold to classify a patient as healthy or as having Wilson disease using dNCC Ratio, the dNCC Ratio of 149 healthy patients and 41 Wilson disease patients was analyzed. Specifically, 60 “Reference” samples and 89 healthy patient samples from Study 104, Study 106 and Study 108 were analyzed. The 41 Wilson disease patients' cohort is from Study 301 and Study 201. The analysis focused on treatment of naïve patients due to potential confounding effects by previous treatment.

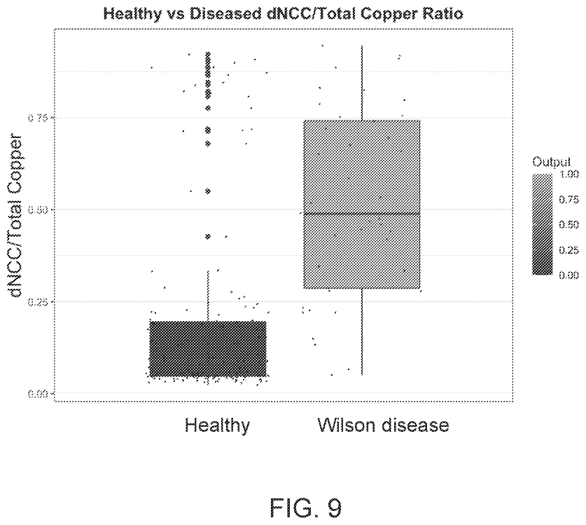

The distribution of healthy vs Wilson disease patients were represented in boxplots (FIG. 9). Using boxplots, the distribution of numerical data and skewness was observed through displaying averages. A T-test was performed to show the significance (p-value <6.082e-10) in the difference of mean of dNCC Ratio between healthy and Wilson disease patients. The performance at each dNCC Ratio data was measured between 0-1 to classify healthy and Wilson disease patients.

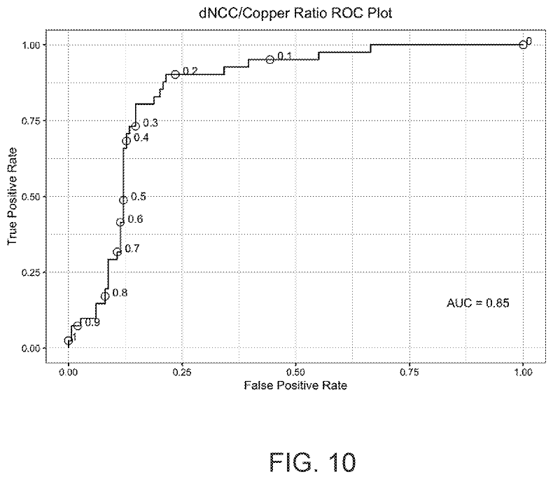

ROCR (Sing T, et al. (2005) ROCR: visualizing classifier performance in R. Bioinformatics 21(20):3940-1) was used, which is a R package to evaluate and a ggplot R package to visualize the performance of this scoring classifier. A receiving operating characteristic (ROC) curve was plotted (FIG. 10) showing the performance of using dNCC Ratio for classification of healthy vs Wilson disease patients. Because the datasets were slightly unbalanced, the F-score was used to define the optimal threshold. In addition, the ROCR tool (Sing T et al. (2005)) was used to measure F-score.

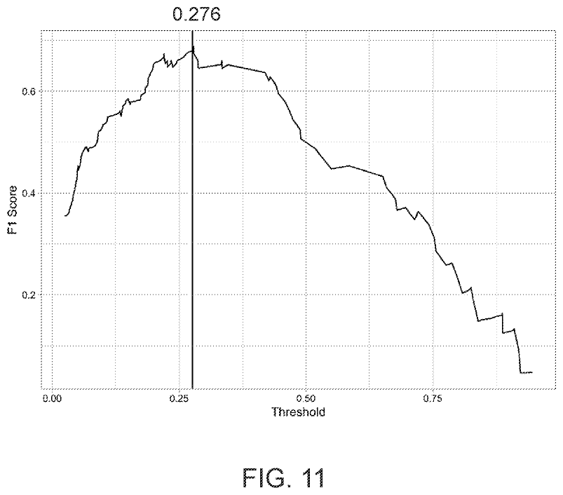

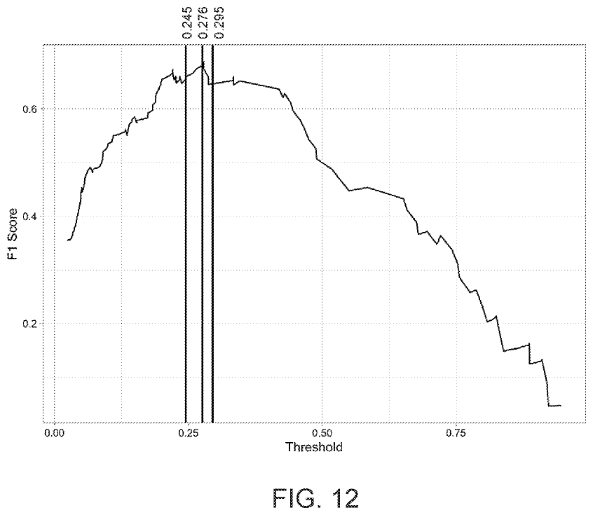

With a F-score maximum at ˜0.69 the optimal threshold of 0.276 or 27.6% of dNCC Ratio was observed (FIG. 11). Considering the specificity and sensitivity trade-offs, a range of thresholds with a F-score above 0.65 were investigated. A range for the dNCC Ratio threshold was defined as shown in FIG. 12 (i.e., from 0.245 to 0.295, inclusive). Finally, the cross-tabulation of observed and predicted classes with associated statistics with confusionMatrix function from Caret (Kuhn. (2008), J. Stat. Soft., Building predictive models in R using caret Package 28; 1-26) library in R was calculated.

Serum and Plasma Sample Comparison

Matched sets of human lithium heparin plasma and serum from 52 healthy individuals were obtained from BIOIVT.

The concentration of LBC in the plasma and serum samples was determined by ICP-MS (Agilent 8900) after performing the validated LBC bioanalytical assay method as described in Example 10 of U.S. Provisional Application No. 62/958,432, filed Jan. 8, 2020, herein incorporated by reference in its entirety, with the anti-CP mAb mixture (1,2,3) disclosed in PCT/US21/49890 replacing the goat anti-human CP antibody.

Briefly, CP was first removed by immunocapture with the anti-CP mAb mixture (1,2,3) to obtain a dNCC fraction, followed by chelation of the dNCC solution with EDTA, and then filtration to collect the labile bound form of copper in the filtrate.

More specifically, about 20 μL of each biological sample were added with about 200 μL beads coated with anti-CP mAb mixture (1,2,3) (˜96 μg total anti-CP mAb per sample) to a well and then subjected to the immunocapture step disclosed in Example 4 of PCT/US21/49890, generating a dNCC fraction per sample.

About 200 μL of the dNCC fraction for each sample was transferred to a clean, metal-free tube, and then about 60 μL of chelation spiking solution (45.5 mM EDTA (Sigma BioUltra) and 456 μM L-Histidine (Sigma BioUltra)) were added to each sample. The samples were gently mixed well and then incubated at approximately 37° C. for about 1 hour. Optionally, the tubes could be centrifuged.

Each incubated sample was transferred to a 2% nitric acid washed 30K MWCO centrifugal filter (regenerated cellulose membrane) (Millipore, AmiconUltra) and centrifuged at approximately 14,000×g for about 35 minutes at about 25° C.

About 200 μL of filtrate were transferred to a new clean, metal-free plastic tube, and about 600 μL of 0.1% HNO₃ in H₂O were added to the metal-free plastic tubes.

About 10 μL of rhodium internal standard spike (100 ng/mL) were added to each of the above metal free tubes. Each tube was then centrifuged at approximately 3500 rpm for about 1 minute and vortexed to mix well.

Quantification of LBC was performed by ICP-MS (Agilent 8900) using rhodium as the internal standard and operating under the conditions and parameters summarized in Tables 5 and 6. A concentric MicroMist nebulizer was used, and the spray chamber temperature was kept at about 2° C. The analysis was performed in He MSMS gas mode.

TABLE 5
ICP-MS Autosampler and Operation Conditions*
Autosampler SPS4

| | |
|---|---|
| Needle rinse 10 s pump speed 0.3 rps | Purified water |
| Wash 1 45 s, pump speed 0.3 rps | 5% HNO₃/H₂O (v/v) |
| Wash 2 45 s, pump speed 0.3 rps | 5% HNO₃/H₂O (v/v) |
| Wash 3 60 s, pump speed 0.3 rps | 0.1% HNO₃/H₂O (v/v) |
| Sample introduction 30 s, pump speed 0.5 rpm | |
| Stabilize 35 s, pump speed 0.1 rps | |

*Conditions may be adjusted to optimize response and minimize carryover.

TABLE 6
ICP-MS Conditions**

| | |
|---|---|
| Mass Spectrometer | Agilent 8900 |
| Nebulizer | Concentric MicroMist nebulizer |
| Spray chamber temp | 4° C. |
| Operation mode | MSMS He gas mode |

Parameters

| | | |
|---|---|---|
| Plasma | RF power (W) | 1550 |
| | Sampling depth (mm) | 10 |
| | Nebulizer gas (L/min) | 1.04 |
| | Nebulizer pump (rps) | 0.10 |
| | Make up gas (L/min) | 0 |
| Cell | He Flow (mL/min) | 5.1 |
| | OctP Bias (V) | −18 |
| | OctP RF (V) | 180 |
| | Energy Discrimination (V) | 0 |
| Lenses | Omega Bias (V) | −120 |
| | Omega Lens (V) | 0 |
| | Cell Entrance (V) | −40 |
| | Cell Exit (V) | −50 |
| | Deflect (V) | −9.0 |
| Integration | Cu 63 | 0.99 sec |
| Time/Mass | Rh 103 (IS) | 0.1 sec |

**Instrument conditions may be adjusted to optimize response.

The ICP-MS system plasma was turned on and “Yes” clicked to perform Auto Tune. Autotune and tune check were performed using a tuning solution (Agilent). The ICP-MS system was equilibrated with the default setting of warming up. The samples were then introduced for ICP-MS measurement.

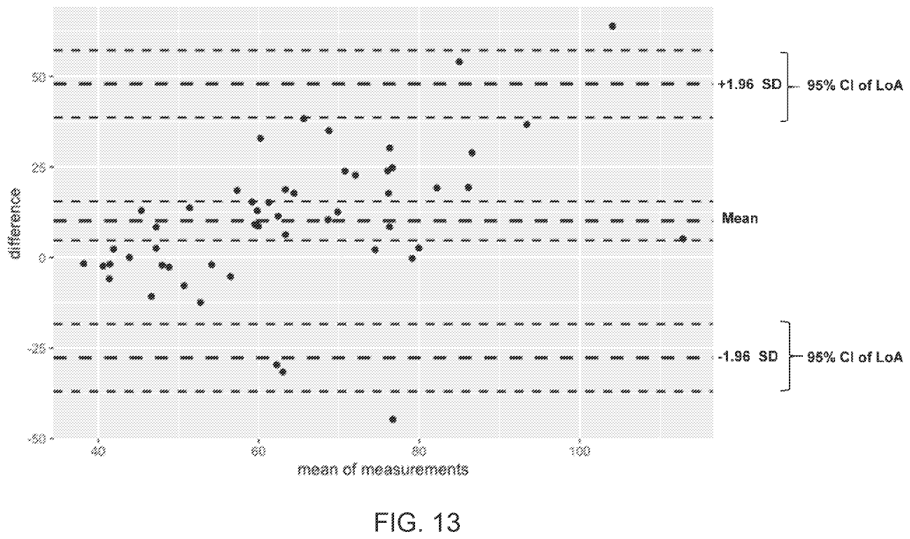

The LBC concentration results in human plasma and serum from the 52 healthy individuals are shown in Table 7. The degree of agreement between the LBC concentrations obtained from human serum and human Li—H plasma from the same sets of 52 heathy individuals was assessed by Bland Altman method (See Giavarnna, Biochimie Medica 2015; 25(2):141-51). FIG. 13 shows the Bland Altman scatter plot with Y axis as the difference between the serum and plasma LBC levels and X axis as the mean of the two. The average of the difference between the two sets of LBC values is 10.1 ng/mL. This means on average the serum LBC value is 10.1 ng/mL higher than plasma LBC.

In the Bland Altman method, it is recommended that 95% of data points lie within ±1.96 standard deviation (SD) of the mean difference, which is defined as the limits of agreement (LOA). In this plot, the upper and lower LOA were calculated as ˜27.7 and 47.9, respectively. The plot also shows the 95% confidence intervals (CI) of the mean difference and of the LOA, which indicate the possible sampling error in the estimate. In summary, two out of 52, accounting for less than 5% of the total sample points, fell outside of the 95% CI of LOA, which may indicate the serum and plasma LBC concentrations agree with each other with a bias of 10.1 ng/mL.

TABLE 7
Determination of LBC Concentrations in Human Lithium
Heparin Plasma and Serum from 52 Healthy Individuals

| | | Serum LBC | Plasma LBC | Average LBC | |
|---|---|---|---|---|---|
| Gender | Age | (ng/mL) | (ng/mL) | (ng/mL) | Difference |
| Female | 61 | 68.85 | 53.63 | 61.24337721 | 15.2 |
| Female | 51 | 66.47 | 60.18 | 63.32487069 | 6.3 |
| Female | 42 | 85.02 | 67.29 | 76.15334185 | 17.7 |
| Female | 36 | 115.46 | 110.28 | 112.87322 | 5.2 |
| Female | 32 | 64.07 | 54.94 | 59.50668676 | 9.1 |
| Female | 24 | 95.79 | 76.45 | 86.12126963 | 19.3 |
| Male | 22 | 80.55 | 72.05 | 76.3015398 | 8.5 |
| Male | 56 | 68.10 | 56.72 | 62.4111332 | 11.4 |
| Male | 54 | 75.54 | 73.44 | 74.49290634 | 2.1 |
| Male | 50 | 64.25 | 55.58 | 59.91582146 | 8.7 |
| Male | 65 | 81.25 | 78.65 | 79.94799549 | 2.6 |
| Male | 35 | 76.12 | 63.56 | 69.84297349 | 12.6 |
| Male | 60 | 47.21 | 78.80 | 63.004 | −31.6 |
| Male | 48 | 46.50 | 58.92 | 52.708 | −12.4 |
| Male | 37 | 38.42 | 44.32 | 41.3695 | −5.9 |
| Male | 42 | 37.31 | 39.02 | 38.161 | −1.7 |
| Male | 66 | 51.40 | 43.01 | 47.2005 | 8.4 |
| Male | 62 | 43.88 | 43.89 | 43.8835 | 0.0 |
| Male | 41 | 47.47 | 50.15 | 48.8095 | −2.7 |
| Male | 59 | 47.42 | 77.02 | 62.2205 | −29.6 |
| Male | 72 | 46.88 | 49.04 | 47.961 | −2.2 |
| Male | 38 | 41.25 | 51.98 | 46.6155 | −10.7 |
| Male | 56 | 83.41 | 60.69 | 72.0485 | 22.7 |
| Male | 31 | 53.82 | 59.10 | 56.4565 | −5.3 |
| Male | 58 | 39.40 | 41.77 | 40.583 | −2.4 |
| Male | 45 | 54.36 | 99.04 | 76.703 | −44.7 |
| Male | 52 | 46.78 | 54.58 | 50.6825 | −7.8 |
| Male | 62 | 40.49 | 42.37 | 41.431 | −1.9 |
| Male | 18 | 43.04 | 40.75 | 41.895 | 2.3 |
| Male | 38 | 58.24 | 44.50 | 51.37 | 13.7 |
| Male | 75 | 66.84 | 51.50 | 59.1695 | 15.3 |
| Male | 55 | 72.68 | 53.95 | 63.314 | 18.7 |
| Male | 63 | 111.98 | 57.99 | 84.984 | 54.0 |
| Male | 62 | 111.75 | 75.05 | 93.398 | 36.7 |
| Male | 56 | 84.75 | 46.44 | 65.5905 | 38.3 |
| Male | 65 | 89.03 | 64.24 | 76.6355 | 24.8 |
| Male | 44 | 91.45 | 61.18 | 76.314 | 30.3 |
| Male | 67 | 91.81 | 72.59 | 82.196 | 19.2 |
| Male | 43 | 73.27 | 55.51 | 64.3895 | 17.8 |
| Male | 64 | 66.54 | 48.04 | 57.2905 | 18.5 |
| Male | 61 | 86.22 | 51.24 | 68.7295 | 35.0 |
| Male | 38 | 101.02 | 72.12 | 86.569 | 28.9 |
| Male | 58 | 76.65 | 43.75 | 60.199 | 32.9 |
| Male | 60 | 88.03 | 64.11 | 76.068 | 23.9 |
| Male | 55 | 78.99 | 79.26 | 79.1245 | −0.3 |
| Male | 38 | 66.25 | 53.33 | 59.7865 | 12.9 |
| Male | 58 | 73.83 | 63.41 | 68.6195 | 10.4 |
| Male | 36 | 136.01 | 72.15 | 104.084 | 63.9 |
| Male | 49 | 82.67 | 58.81 | 70.7415 | 23.9 |
| Male | 23 | 53.13 | 55.11 | 54.124 | −2.0 |
| Male | 34 | 51.84 | 38.90 | 45.371 | 12.9 |
| Male | 55 | 48.47 | 45.96 | 47.2105 | 2.5 |

| | |
|---|---|
| Mean Difference | 10.1 |
| SD | 19.3 |
| Alpha | 0.05 |
| 95% Lower LOA | −27.7 |
| 95% Upper LOA | 47.9 |

It is understood that the examples and embodiments described herein are for illustrative purposes only and that various modifications or changes in light thereof are suggested to persons skilled in the art and are to be incorporated within the spirit and purview of this application and scope of the appended claims. All publications, patents, and patent applications cited herein are hereby incorporated herein by reference for all purposes.

What is claimed is:

1. A method for treating a copper metabolism-associated disease or disorder in a subject, the method comprising:
determining a concentration of total copper and a concentration of labile-bound copper (LBC) in the subject's biological sample;
determining the ratio of LBC to total copper in the subject's biological sample; and
administering to the subject a therapeutically effective amount of bis-choline tetrathiomolybdate when the ratio of LBC to total copper in the subject's biological sample is ≥ between 0.21 and 0.27.

2. The method of claim 1, wherein the bis-choline tetrathiomolybdate is administered to the subject when the ratio of LBC to total copper in the subject's biological sample is ≥0.21.

3. The method of claim 1, wherein the bis-choline tetrathiomolybdate is administered to the subject when the ratio of LBC to total copper in the subject's biological sample is ≥0.24.

4. The method of claim 1, wherein the bis-choline tetrathiomolybdate is administered to the subject when the ratio of LBC to total copper in the subject's biological sample is ≥0.27.

5. The method of any one of claims 1 to 4, wherein the copper metabolism-associated disease or disorder is Wilson disease.

6. The method of any one of claims 1 to 5, wherein the concentration of total copper in the subject's biological sample is measured by inductively coupled plasma-mass spectrometry (ICP-MS).

7. The method of any one of claims 1 to 6, wherein the concentration of LBC in the subject's biological sample is determined using an LBC assay.

8. The method of any one of claims 1 to 7, wherein the subject previously received no treatment for the copper metabolism-associated disease or disorder, such as for Wilson disease (i.e., a treatment-naïve subject).

9. The method of any one of claims 1 to 8, wherein the therapeutically effective amount of bis-choline tetrathiomolybdate is in the range of about 15 mg to about 60 mg per day.

10. The method of any one of claims 1 to 8, wherein the therapeutically effective amount of bis-choline tetrathiomolybdate is about 15 mg daily.

11. The method of any one of claims 1 to 8, wherein the therapeutically effective amount of bis-choline tetrathiomolybdate is about 30 mg daily (e.g., 2×15 mg daily).

12. The method of any one of claims 1 to 8, wherein the therapeutically effective amount of bis-choline tetrathiomolybdate is about 60 mg daily (e.g., 4×15 mg daily).

13. The method of any one of claims 1 to 12, wherein, following the administration of the therapeutically effective amount of bis-choline tetrathiomolybdate to the subject, the subject shows an improvement in disability status, psychiatric symptoms, clinical symptoms, or treatment satisfaction.

14. The method of any one of claims 1 to 13, wherein the biological sample comprises human plasma or human serum.

15. The method of any one of claims 1 to 14, wherein the biological sample comprises human plasma.

16. The method of any one of claims 1 to 14, wherein the biological sample comprises human serum.

17. A method of diagnosing a copper metabolism-associated disease or disorder in a subject, the method comprising:
determining a concentration of total copper and a concentration of LBC in the subject's biological sample;
determining the ratio of LBC to total copper in the subject's biological sample; and
diagnosing the subject with a copper metabolism-associated disease or disorder if the ratio of LBC to total copper in the subject's biological sample is ≥ between 0.21 and 0.27.

18. The method of claim 17, wherein the subject is diagnosed with the copper metabolism-associated disease or disorder when the ratio of LBC to total copper in the subject's biological sample is ≥0.21.

19. The method of claim 17, wherein the subject is diagnosed with the copper metabolism-associated disease or disorder when the ratio of LBC to total copper in the subject's biological sample is ≥0.24.

20. The method of claim 17, wherein the subject is diagnosed with the copper metabolism-associated disease or disorder when the ratio of LBC to total copper in the subject's biological sample is ≥0.27.

21. The method of any one of claims 17 to 20, wherein the copper metabolism-associated disease or disorder is Wilson disease.

22. The method of any one of claims 17 to 21, wherein the concentration of total copper in the subject's biological sample is measured by inductively coupled plasma-mass spectrometry (ICP-MS).

23. The method of any one of claims 17 to 22, wherein the concentration of LBC in the subject's biological sample is determined using an LBC assay.

24. The method of any one of claims 17 to 23, wherein the biological sample comprises human plasma or human serum.

25. The method of any one of claims 17 to 24, wherein the biological sample comprises human plasma.

26. The method of any one of claims 17 to 24, wherein the biological sample comprises human serum.

27. A method of identifying a subject as suited for treatment with bis-choline tetrathiomolybdate, the method comprising:
determining a concentration of total copper and a concentration of labile-bound copper (LBC) in the subject's biological sample;
determining the ratio of LBC to total copper in the subject's biological sample;
identifying the subject as suited for treatment with bis-choline tetrathiomolybdate when the ratio of LBC to total copper in the subject's biological sample is ≥ between 0.21 and 0.27, and
optionally administering a therapeutically effective amount of bis-choline tetrathiomolybdate to the subject identified as suited for treatment with bis-choline tetrathiomolybdate.

28. The method of claim 27, wherein the subject is identified as suited for treatment with bis-choline tetrathiomolybdate when the ratio of LBC to total copper in the subject's biological sample is ≥0.21.

29. The method of claim 27, wherein the subject is identified as suited for treatment with bis-choline tetrathiomolybdate when the ratio of LBC to total copper in the subject's biological sample is ≥0.24.

30. The method of claim 27, wherein the subject is identified as suited for treatment with bis-choline tetrathiomolybdate when the ratio of LBC to total copper in the subject's biological sample is ≥0.27.

31. The method of any one of claims 27 to 30, wherein the concentration of total copper in the subject's biological sample is measured by inductively coupled plasma-mass spectrometry (ICP-MS).

32. The method of any one of claims 27 to 31, wherein the concentration of LBC in the subject's biological sample is determined using an LBC assay.

33. The method of any one of claims 27 to 32, wherein the therapeutically effective amount of bis-choline tetrathiomolybdate is in the range of about 15 mg to about 60 mg per day.

34. The method of any one of claims 27 to 32, wherein the therapeutically effective amount of bis-choline tetrathiomolybdate is about 15 mg daily.

35. The method of any one of claims 27 to 32, wherein the therapeutically effective amount of bis-choline tetrathiomolybdate is about 30 mg daily (e.g., 2×15 mg daily).

36. The method of any one of claims 27 to 32, wherein the therapeutically effective amount of bis-choline tetrathiomolybdate is about 60 mg daily (e.g., 4×15 mg daily).

37. The method of any one of claims 27 to 36, wherein, following the optional administration of the therapeutically effective amount of bis-choline tetrathiomolybdate to the subject, the subject shows an improvement in disability status, psychiatric symptoms, clinical symptoms, or treatment satisfaction.

38. The method of any one of claims 27 to 37, wherein the biological sample comprises human plasma or human serum.

39. The method of any one of claims 27 to 38, wherein the biological sample comprises human plasma.

40. The method of any one of claims 27 to 38, wherein the biological sample comprises human serum.

41. A method for treating a copper metabolism-associated disease or disorder in a subject, the method comprising:
determining a concentration of total copper and a concentration of directly measured non-ceruloplasmin-bound copper (dNCC) in the subject's biological sample;
determining the ratio of dNCC to total copper in the subject's biological sample; and
administering to the subject a therapeutically effective amount of bis-choline tetrathiomolybdate when the ratio of dNCC to total copper in the subject's biological sample is ≥ between 0.245 and 0.295.

42. The method of claim 41, wherein the bis-choline tetrathiomolybdate is administered to the subject when the ratio of dNCC to total copper in the subject's biological sample is ≥0.2456.

43. The method of claim 41, wherein the bis-choline tetrathiomolybdate is administered to the subject when the ratio of dNCC to total copper in the subject's biological sample is ≥0.276.

44. The method of claim 41, wherein the bis-choline tetrathiomolybdate is administered to the subject when the ratio of dNCC to total copper in the subject's biological sample is a 0.295.

45. The method of any one of claims 41 to 44, wherein the therapeutically effective amount of bis-choline tetrathiomolybdate is in the range of about 15 mg to about 60 mg per day.

46. The method of any one of claims 41 to 44, wherein the therapeutically effective amount of bis-choline tetrathiomolybdate is about 15 mg daily.

47. The method of any one of claims 41 to 44, wherein the therapeutically effective amount of bis-choline tetrathiomolybdate is about 30 mg daily (e.g., 2×15 mg daily).

48. The method of any one of claims 41 to 44, wherein the therapeutically effective amount of bis-choline tetrathiomolybdate is about 60 mg daily (e.g., 4×15 mg daily).

49. The method of any one of claims 41 to 48, wherein, following the administration of the therapeutically effective amount of bis-choline tetrathiomolybdate to the subject, the subject shows an improvement in disability status, psychiatric symptoms, clinical symptoms, or treatment satisfaction.

50. A method of diagnosing a copper metabolism-associated disease or disorder in a subject, the method comprising:
determining a concentration of total copper and a concentration of dNCC in the subject's biological sample;
determining the ratio of dNCC to total copper in the subject's biological sample; and
diagnosing the subject with a copper metabolism-associated disease or disorder if the ratio of dNCC to total copper in the subject's biological sample is ≥ between 0.245 and 0.295.

51. The method of claim 50, wherein the subject is diagnosed with the copper metabolism-associated disease or disorder when the ratio of dNCC to total copper in the subject's biological sample is ≥0.2456.

52. The method of claim 50, wherein the subject is diagnosed with the copper metabolism-associated disease or disorder when the ratio of dNCC to total copper in the subject's biological sample is ≥0.276.

53. The method of claim 50, wherein the subject is diagnosed with the copper metabolism-associated disease or disorder when the ratio of dNCC to total copper in the subject's biological sample is ≥0.295.

54. The method of any one of claims 41 to 53, wherein the copper metabolism-associated disease or disorder is Wilson disease.

55. The method of any one of claims 41 to 54, wherein the concentration of total copper in the subject's biological sample is measured by inductively coupled plasma-mass spectrometry (ICP-MS).

56. The method of any one of claims 41 to 54, wherein the concentration of dNCC in the subject's biological sample is determined using a dNCC assay.

57. The method of any one of claims 41 to 56, wherein the subject previously received no treatment for the copper metabolism-associated disease or disorder, such as for Wilson disease (i.e., a treatment-naïve subject).

58. The method of any one of claims 41 to 57, wherein the biological sample comprises human plasma or human serum.

59. The method of any one of claims 41 to 58, wherein the biological sample comprises human plasma.

60. The method of any one of claims 41 to 58, wherein the biological sample comprises human serum.

61. A method of identifying a subject as suited for treatment with bis-choline tetrathiomolybdate, the method comprising:
determining a concentration of total copper and a concentration of dNCC in the subject's biological sample;
determining the ratio of dNCC to total copper in the subject's biological sample;
identifying the subject as suited for treatment with bis-choline tetrathiomolybdate when the ratio of dNCC to total copper in the subject's biological sample is ≥ between 0.245 and 0.295, and
optionally administering a therapeutically effective amount of bis-choline tetrathiomolybdate to the subject identified as suited for treatment with bis-choline tetrathiomolybdate.

62. The method of claim 61, wherein the subject is identified as suited for treatment with bis-choline tetrathiomolybdate when the ratio of dNCC to total copper in the subject's biological sample is ≥0.245.

63. The method of claim 61, wherein the subject is identified as suited for treatment with bis-choline tetrathiomolybdate when the ratio of dNCC to total copper in the subject's biological sample is ≥0.276.

64. The method of claim 61, wherein the subject is identified as suited for treatment with bis-choline tetrathiomolybdate when the ratio of dNCC to total copper in the subject's biological sample is ≥0.295.

65. The method of any one of claims 61 to 64, wherein the concentration of total copper in the subject's biological sample is measured by inductively coupled plasma-mass spectrometry (ICP-MS).

66. The method of any one of claims 61 to 65, wherein the concentration of dNCC in the subject's biological sample is determined using a dNCC assay.

67. The method of any one of claims 61 to 66, wherein the therapeutically effective amount of bis-choline tetrathiomolybdate is in the range of about 15 mg to about 60 mg per day.

68. The method of any one of claims 61 to 66, wherein the therapeutically effective amount of bis-choline tetrathiomolybdate is about 15 mg daily.

69. The method of any one of claims 61 to 66, wherein the therapeutically effective amount of bis-choline tetrathiomolybdate is about 30 mg daily (e.g., 2×15 mg daily).

70. The method of any one of claims 61 to 66, wherein the therapeutically effective amount of bis-choline tetrathiomolybdate is about 60 mg daily (e.g., 4×15 mg daily).

71. The method of any one of claims 61 to 70, wherein, following the optional administration of the therapeutically effective amount of bis-choline tetrathiomolybdate to the subject, the subject shows an improvement in disability status, psychiatric symptoms, clinical symptoms, or treatment satisfaction.

72. The method of any one of claims 61 to 71, wherein the biological sample comprises human plasma or human serum.

73. The method of any one of claims 61 to 72, wherein the biological sample comprises human plasma.

74. The method of any one of claims 61 to 72, wherein the biological sample comprises human serum.
